(19) United States
(12) Patent Application Publication
(10) Pub. No.: US 2024/0377392 A1
(43) Pub. Date: Nov. 14, 2024

(54) MODULAR TEST SWAB CONTROL SYSTEM FOR LATERAL FLOW TESTS

(71) Applicant: The United States of America, as Represented by the Secretary of Agriculture, Washington, DC (US)

(72) Inventors: Robert M. Hnasko, Albany, CA (US); Ronald P. Haff, Albany, CA (US); Jeffery A. McGarvey, Albany, CA (US); Alice V. Lin, Albany, CA (US); Eric S. Jackson, Albany, CA (US)

(21) Appl. No.: 18/316,082

(22) Filed: May 11, 2023

(51) Int. Cl. G01N 33/543

(52) U.S. Cl. CPC G01N 33/54388

(57) ABSTRACT

A modular biosensor platform is designed to perform a rapid lateral flow immunoassay. The system includes a swab retention module and a lateral flow cassette module to enable users to perform simple point-of-care (POC) tests in a portable setting. The system includes the necessary assay components to detect target analytes collected on a fiber swab. The system is configured so that a test result is visible on an immunochromatographic test strip that can be viewed through a “results” window in the system's lateral flow cassette module.

### FIELD OF THE INVENTION

The modular test swab control system relates to a single-use biosensor platform designed to perform rapid lateral flow immunoassay (LFIA)-type tests or other swab-based tests. The system uses modular components that enable users to perform simple point-of-care (POC) tests in a portable setting. Specifically, the system described herein comprises a biosensor platform that fully integrates all the necessary LIFA components for the rapid detection of target analytes that are collected on a fiber swab. The system is configured so that a test result is visible on an immunochromatographic test strip that is visible through a “results” window in the system.

### BACKGROUND OF THE INVENTION

There is an expanding demand for rapid POC testing in the general health and food safety areas. These types of tests can provide inexpensive and easy-to-use detection options for allergens, pathogens, toxins, adulterants and other environmental contaminants from the farm to the fork. Based on current data, the market for these types of tests is projected to expand at a rate of about 10% annually—and eventually grow to more than $38 billion. North America accounts for the greatest share of the market at a projected market size of $16B, with the largest growth occurring in lateral flow assays, detection of infectious diseases, and ‘at home’ and personal end-user health management segments.

The most successful lateral flow devices (to date) include detection of human chorionic gonadotropin (hCG), a hormone associated with human pregnancy, and assays that detect the presence of specific (usually illicit) drugs in the system of a test subject. These assays utilize direct liquid urine samples and consequently device designs do not require a liquid sample extraction buffer and a precision liquid delivery mechanism.

Many applicable tests, particularly in agriculture, require the use of a fibrous swab as a means of collecting a sample. One end of the swab is typically comprised of an absorptive fibrous head (such as a cotton swab) connected to a thin wooden or plastic stem. The absorptive end can be used to harvest sample material for testing, such as nasopharyngeal, oral, or rectal testable sample material. After collection, the sample swab is transferred to a secondary container for recovery of the biological material and downstream testing.

The current state-of-the-art lateral flow devices provide separate poorly integrated components that rely on multi-step procedures to perform simple tests. End-user error is a significant problem. Multi-step science-kit methodologies are frequently impractical, prone to error, cumbersome to perform, and less desirable to end-users in field locations.

The need exists for simple modular test systems for lateral flow immunoassay (LFIA)-type tests. The current invention comprises a novel swab control module for rapid interface with a modular biosensor cassette that holds a LIFA immunochromatographic test strip. The current biosensor platform provides users with rapid and accurate test results in a single step with minimal end-user training. The inventors' flexible biosensor platforms comprise compact field-portable units that provide a stand-alone test that includes fully integrated sample extraction capability and liquid delivery to immunochromatographic test strips.

Components of the modular biosensor platforms described herein are designed for ease of manufacturing and assembly. The standardized module parts are generally interchangeable to accommodate different test and liquid extraction/buffer combinations. The inventors' modular biosensor platforms are designed so that the platforms may be 3-D printed for small-scale testing or injection molded for large-scale production and injection molded for large scale production. Additionally, the modular platforms are compatible with a separate digital reader tool that allows rapid digital porting of test strip results onto a data platform for recording and analyzing the resulting test data.

### SUMMARY OF THE INVENTION

This disclosure is directed to a modular test swab control system 10 comprising a test swab retention module 20 that is selectively attached to a lateral flow cassette module 40.

The test swab retention module 20 comprises at least a test swab receiver assembly 24 that is configured to hold and retain a test swab head 14. The test swab retention module 20 includes a lid 22 that can be selectively closed to protect the test swab head 14 and secure the test swab 12 in the test swab receiver 24. The test swab retention module 20 also includes a liquid-retaining reservoir component (i.e. a “test swab seat”) 26 that slides into the receiver 24 so that sample-retaining liquid is optionally retained within the test swab retention module 20. The test swab seat 26 is ramped so liquid will flow down away from the sample swab head and into the port 36. Temporal liquid retention in the swab module 20 is optional with use of a frangible seal 38. These alternating ramps 24, 26, 46a along with ports 36 and 46 provide a means to trap solid contaminants and modulate the speed and volume of liquid delivery to the test strip 43 sample pad 47.

A lateral flow cassette module 40 is selectively attached to the test swab retention module 20. The lateral flow cassette module 40 comprises at least a top plate 42 having a test results window 48 and a base plate 44 comprising an overflow reservoir 51. A horizontally oriented LIFA immunochromatographic test strip 43 is sandwiched between the top plate 42 and the base plate 44 of the lateral flow cassette module 40.

To conduct a LIFA test, in one preferred embodiment, a test swab 12 is inserted into the test swab receiver 24 and the test swab head 14 is hydrated with a carrier liquid so that sample material is entrained in the carrier liquid. The sample-entraining liquid flows from the test swab head 14 down through the test swab receiver 24 and the test swab seat 26 to the immunochromatographic test strip 43 in the lateral flow cassette module 40. When the LIFA test is complete, a test result is visible through the test results window 48 in the lateral flow cassette module 40.

### BRIEF DESCRIPTION OF THE DRAWINGS

FIG. 1 is a top elevational perspective view of a modular swab control system 10 as described herein. FIG. 1 shows a typical test swab 12 inserted in the modular swab control system 10.

FIG. 2 is an exploded view of the swab control system 10 shown in FIG. 1. As shown in FIG. 2, the system 10 comprises a swab retention module 20 and a lateral flow cassette module 40. FIG. 2 shows the assembled modular swab control system 10 without a test swab 12 present in the test swab aperture 34.

FIG. 3 is a bottom exploded view of the swab retention module 20.

FIG. 4 is a top exploded view of the two-part lateral flow cassette module 40 including the top 42 and base 44 plates. FIG. 4 also shows an immunochromatographic test strip 43 as it would be positioned (in operation) in the lateral flow cassette module 40.

FIG. 5 is an exploded view of an alternative embodiment showing the bottom of the test swab retention module 20 with a frangible cover 38 positioned over the outlet port opening 36, as it connects with the (top view of the) top plate 42 of the two-part lateral flow cassette module 40.

Note that assemblies/systems in some of the FIGs. may contain multiple examples of essentially the same component. For simplicity and clarity, only a small number of the example components may be identified with a reference number. Unless otherwise specified, other non-referenced components with essentially the same structure as the exemplary component should be considered to be identified by the same reference number as the exemplary component. Further, unless specifically indicated otherwise, drawing components may or may not be shown to scale.

### DETAILED DESCRIPTION OF PREFERRED EMBODIMENTS

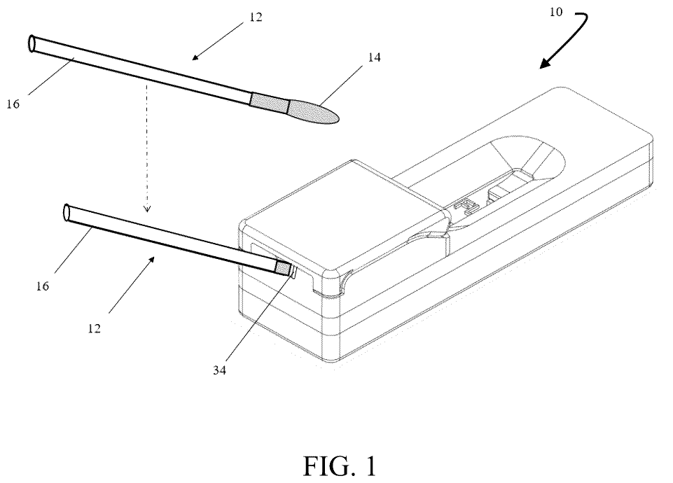

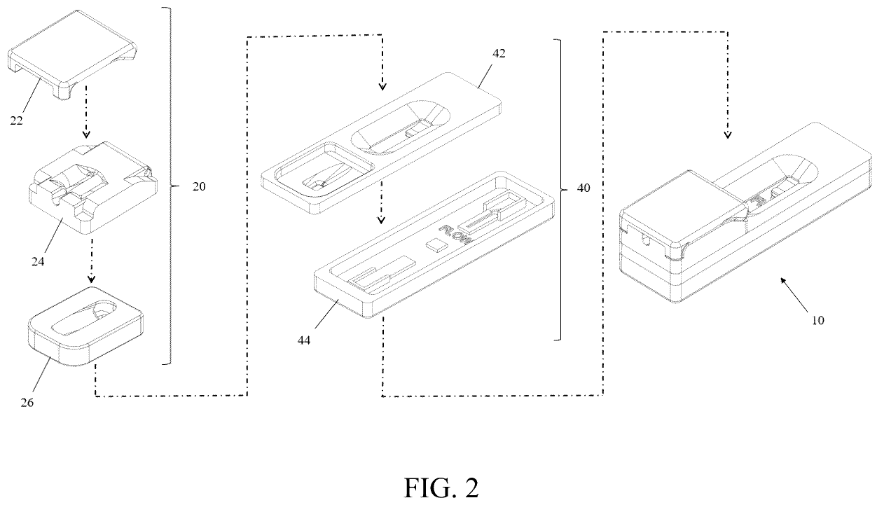

As generally shown in FIG. 1, the current disclosure describes a modular swab control system 10. FIG. 1 shows an exemplary test swab 12 that has a preferably fibrous head/tip 14 and an elongated stem 16. In the preferred embodiment, the test swab 12 comprises a cotton head 14. In alternative embodiments, the test swab head 12 may comprise any material known in the art consistent with retaining a testable material/sample. FIG. 1 further shows the test swab 12 inserted into a swab insertion slot 34 of the modular swab control system 10. As best shown in FIG. 2, the system 10 comprises a swab retention module 20 and lateral flow cassette module 40.

As shown in FIG. 2, the swab retention module 20 comprises a retainer lid 22 that covers and at least partially encloses a swab receiver assembly 24. The swab receiver assembly 24 is sandwiched between the retainer lid 22 and a swab seat 26 so that the swab receiver 24 slides over and partially encloses the swab seat 26.

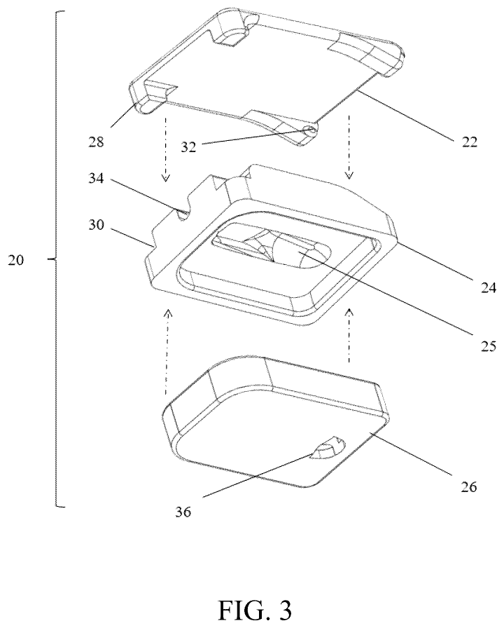

FIG. 3 shows a bottom view of the swab retention module 20 in greater detail. As shown in FIG. 3, the swab receiver 24 further comprises a hood 25 with a concave inner surface that cradles the test swab head 14. For the purposes of this disclosure, the term “cradles” is defined as at least partially enclosing the swab head and holding the swab head in place.

Once the swab head 14 is inserted in the swab receiver 24, the test swab head 14 is held in place by the test swab insertion slot 34, and the test swab receiver hood 25, and further protected by the lid 22. Once the swab head is fully inserted, downward pressure on the swab stem 16 will not cause the swab head 14 to pop up and out of the swab retention module 20. Downward movement of the swab head 14 is prevented by the swab seat 26.

As shown in FIG. 3, the front of the swab retention module retainer lid 22 comprises a pair of retention teeth 28 that snap into corresponding grooves 30 in the swab receiver assembly 24. A pair of bilateral seat pinpoints 32 in the rear of the retention lid 22 act as hinges and snap into corresponding grooves in the rear of the swab receiver 24 so that the retainer lid 22 is hinged to the swab receiver assembly 24 thereby enabling the retainer lid 22 to be selectively opened and closed to insert or remove a test swab head 14. The retainer lid 22 serves to: (a) protect the swab head 14; (b) limit cross contact contamination of the sample; (c) provide a means to secure sample integrity; and, (d) provide an optimal (top outer) surface for the placement of a sample identification label.

As shown in FIG. 3, the swab receiver insertion slot 34 is angled downwardly to create a microliquidic pathway so that carrier liquid applied to test swab head 14 entrains a portion of the sample from the test swab head 14 and flows through the swab receiver 24 and into the swab receiver seat 26. As shown in FIG. 3, the swab receiver seat 26 slides up into the base of the swab receiver 24. The bottom/floor of the swab receiver seat 26 is angled so that the sample-entraining liquid is ultimately directed to an outlet port opening 36 at the bottom of the swab receiver seat 26.

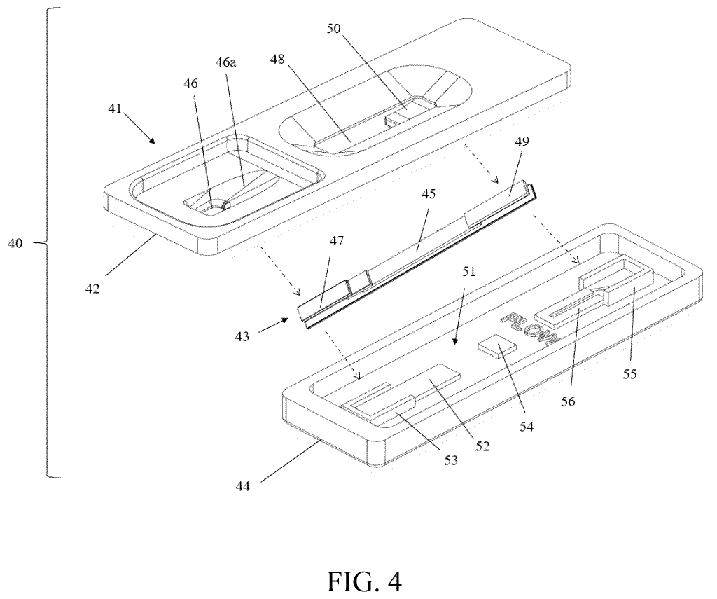

As best shown in FIG. 4, the lateral flow cassette module 40 comprises a top plate 42 and a base plate 44. Sample-entraining liquid from the swab retention module outlet port 36 is directed to a receiving end 41 of the top plate 42. The sample-entraining liquid is then funneled through a receiving aperture 46 in the top plate 42 and down a ramp 46a and into to the base plate 44. The ramp 46a directs liquid from swab module port 36 toward the port opening 46 of lateral flow cassette module 40 where it contacts the lateral flow test strip 43 sample pad 47 and initiates capillary fluid flow through the various test strip 43 materials 47, 45, 49.

As generally shown in FIG. 4, in operation, a lateral flow immunoassay (LFIA) immunochromatographic test strip 43 is sandwiched between the lateral flow cassette module top plate 42 and the base plate 44. The “test results” portion 45 of the test strip 43 is visible through a “test results window” 48 in the top plate 42 of the cassette module 40.

For the purposes of this disclosure, the “test results” portion of the test strip 43 is an intermediate portion of the test strip 43 associated with at least one symbol or other visual indicator that reveals the results of a test. A “test results window” is defined as an aperture in the top plate 42 of the cassette module 40 that enables a user to view the “test results” portion of the immunochromatographic test strip 43.

The test strip 43 is held in place by a resilient flex plate 50 that extends diagonally downward from the top plate 42. For the purposes of this disclosure, the term “resilient” means capable of bending/flexing significantly without breaking. In the preferred embodiment, the flex plate 50 sandwiches/secures the “terminal” portion of the test strip 43 between the flex plate 50 and a raised terminal platform 56 extending upwardly from a floor 51 of the base plate 44.

As shown in FIG. 4, the base plate 44 comprises a plurality of raised platforms 52, 54, 56 that support the LIFA immunochromatographic test strip 43 and raise the test strip 43 above the floor 51 of the base plate 44. The enclosed bottom/floor 51 of the base plate 44 essentially comprises an overflow liquid-retaining reservoir for sample-entraining liquid that is not absorbed/retained by the immunochromatographic test strip 43.

As shown in FIG. 4, the first raised platform (the “sample pad” platform) 52 supports the sample pad 47 portion of the test strip 43. A raised collar 53 around the first platform 52 ensures that the sample pad 47 portion of the test strip 43 is saturated by the incoming sample-entraining liquid before any excess liquid flows down to the overflow reservoir/floor 51 of the base plate 44. The second raised platform (the “intermediate” platform) 54 supports the intermediate section 45 (including the “results” section) of the test strip 43, and the third raised platform (the “terminal” platform) 56 supports the terminal portion 49 of the test strip 43. A raised collar 55 is also positioned around the terminal platform 56.

In operation, to initiate a test, a biological sample is collected on the fibrous head 14 of a test swab 12. The swab retainer lid 22 is opened and the test swab head 14 is positioned in the swab receiver 24 so that the swab stem 16 extends outwardly through the swab insertion slot 34 of the receiver assembly 24.

Once the test swab 12 is in position in the receiver assembly 24, a liquid buffer(s) (and/or other chemicals or carrier agents) are directly applied to the swab head 14. Note that, for the purposes of this disclosure, the terms “carrier liquid” and “buffering liquid” are used interchangeably to mean “carrier liquid and/or buffering liquid (including water) or other chemical substances or solvents in a liquid form”. The test swab head 14 (via the test swab stem 16) may be rotated several times during hydration to facilitate the entrainment of the sample material in the applied carrier liquid. Once the swab head 14 is fully hydrated, the lid 22 may be closed and the test swab stem 16 may be broken, cut, or otherwise disconnected from the swab head 14 so that only the swab head 14 is retained within the swab retention module 20.

After the swab head 14 is hydrated, the sample-entraining liquid flows through the swab receiver assembly 24 and into the swab seat 26. The sample-entraining liquid then preferably flows out of the swab seat outlet port 36 and into the lateral flow cassette module 40.

Specifically, as shown in FIG. 4, the sample-entraining liquid flows through the liquid receiving aperture 46 in the top plate 42 of the cassette module 40 and is directed down ramp 46a and to the sample pad portion 47 of the of the LFIA immunochromatographic test strip 43. Preferably the sample-entraining liquid fully irrigates and saturates the sample pad portion 47 of the test strip 43.

The sample-entraining liquid is then wicked via capillary action through one or more contiguous sections of the test strip 43. As the sample-entraining liquid migrates through the sections of the test strip 43, the liquid may be combined or treated with reagents or other chemicals impregnated in the fibers/fabric of the test strip 43.

The sample-entrained test liquid eventually migrates to an intermediate “results” section 45 of the test strip 43. The test strip results section 45 typically includes a chemically sensitive color/line/symbol indicator on the test strip 43 that reveals the result of the test. The top plate 42 of cassette module 40 is structured so that the test result is visible through a “test results window” 48.

Ultimately the sample-entraining liquid migrates to a “terminal” section 49 of the strip 43. A series of raised platforms 52, 54, 56 extending upwardly from the floor 51 of the base plate 44 support and elevate the test strip 43 during the test process. Raising the test strip 43 above the base plate floor 51 ensures that any excess sample-entraining liquid is drained away from the test strip 43 and does not interfere with the test.

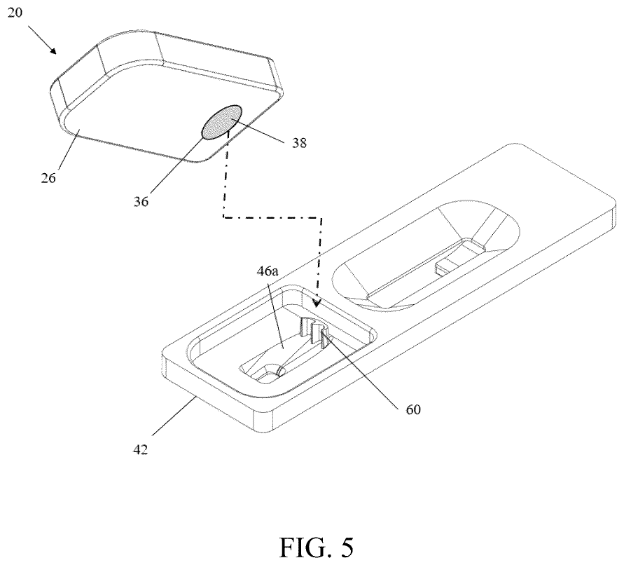

In an alternative embodiment shown in FIG. 5, the test swab retention module 20 may be disconnected from the lateral flow cassette module 40 and the outlet port opening 36 in the swab receiver seat 26 may be covered/sealed by a frangible membrane 38 so that the swab retention module 20 comprises a stand-alone sample storage container. In this embodiment, the top plate 42 of the cassette module 40 includes a set of upwardly extending “teeth” 60 or other means (spikes, blades, etc.) of puncturing the frangible membrane 38 covering the outlet port opening 36. The teeth 60 can be adjusted in size or number to influence the opening size of the frangible membrane 38 and rate of fluid flow through the port 36.

As shown in FIG. 5, in operation, the stand-alone/storage container embodiment of the swab retaining module 20 may be integrated with the cassette module 40 by snapping the retaining module 20 on to the cassette module 40 so that the frangible membrane 38 is punctured by the teeth 60 extending upwardly from the top plate 42 of the cassette module 40. After the frangible seal 38 is punctured, the sample-entraining liquid flows into the cassette module 40 and the test proceeds as described supra.

All components of the modular swab control system 10 are 3-D printable or injection mouldable. The system 10 is energy independent, traceable, and can be adapted to a microelectronic reader for digital interrogation of results.

For the foregoing reasons, it is clear that the subject matter described herein provides an innovative modular swab control system for lateral flow immunoassay tests that may be used in multiple standardized test applications. The current system may be modified in multiple ways and applied in various technological applications. For example, although the preferred embodiment of the modular swab control system is directed to biological tests, the system may also be used for non-biological tests. The disclosed method and apparatus may be modified and customized as required by a specific operation or application, and the individual components may be modified and defined, as required, to achieve the desired result.

Although the materials of construction are not described, they may include a variety of compositions consistent with the function described herein. Such variations are not to be regarded as a departure from the spirit and scope of this disclosure, and all such modifications as would be obvious to one skilled in the art are intended to be included within the scope of the following claims.

The amounts, percentages and ranges disclosed in this specification are not meant to be limiting, and increments between the recited amounts, percentages and ranges are specifically envisioned as part of the invention. All ranges and parameters disclosed herein are understood to encompass any and all sub-ranges subsumed therein, and every number between the endpoints. For example, a stated range of “1 to 10” should be considered to include any and all sub-ranges between (and inclusive of) the minimum value of 1 and the maximum value of 10 including all integer values and decimal values; that is, all sub-ranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions, and so forth as used in the specification and claims are to be understood as being modified in all instances by the implied term “about.” The (stated or implied) term “about” indicates that a numerically quantifiable measurement is assumed to vary by as much as 30 percent, but preferably by as much as 10%. Essentially, as used herein, the term “about” refers to a quantity, level, value, or amount that varies by as much 10% to a reference quantity, level, value, or amount. Accordingly, unless otherwise indicated, the numerical properties set forth in the following specification and claims are approximations that may vary depending on the desired properties sought to be obtained in embodiments of the present invention.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein). The invention illustratively disclosed herein suitably may be practiced in the absence of any element which is not specifically disclosed herein. The term “an effective amount” as applied to a component or a function excludes trace amounts of the component, or the presence of a component or a function in a form or a way that one of ordinary skill would consider not to have a material effect on an associated product or process.

What is claimed is:

1. A modular test swab control system, the system comprising:
a test swab retention module comprising:
a test swab-retaining lid selectively covering a test swab head; and,
a test swab receiver assembly configured to hold and retain the test swab head;
a lateral flow cassette module selectively attached to the test swab retention module, the lateral flow cassette module comprising:
a top plate having a test results window;
a base plate comprising an overflow reservoir, and,
a horizontally oriented lateral flow immunoassay immunochromatographic test strip sandwiched between the top plate and the base plate;
wherein the system is structured so when the test swab head is hydrated, sample-entraining liquid flows from the test swab head down through the test swab receiver to the immunochromatographic test strip so that a test result is visible through the test results window in the top plate.

2. The system of claim 1 wherein the system further comprises a test swab seat connected to the test swab receiver assembly so that the test swab receiver assembly is sandwiched between the test swab retaining lid and the test swab seat.

3. The system of claim 2 wherein the test swab seat slides into the test swab receiver so that the test swab receiver at least partially encloses the test swab seat.

4. The system of claim 2 wherein the test swab seat further comprises an outlet port so that the sample-entraining liquid flows from the test swab head, through the test swab receiver, and out of the test swab seat outlet port, and then down to the immunochromatographic test strip.

5. The system of claim 4 wherein the test swab seat outlet port includes a frangible membrane so that before the test swab retention module is selectively connected to the lateral flow cassette module, the sample-entraining liquid is contained within the test swab seat so that the test swab retention module comprises a stand-alone sample-entraining liquid storage container.

6. The system of claim 5 wherein the top plate of the of the lateral flow module further comprises upwardly extending teeth;
wherein the system is structured so that when the test swab module is snapped onto the lateral flow cassette module, the teeth puncture the frangible membrane and the sample-entraining liquid flows into the lateral flow cassette module to the immunochromatographic test strip.

7. The system of claim 1 wherein the test swab retaining lid comprises a hinged lid that is configured to be selectively opened to facilitate placement of the test swab head, and then the test swab retaining lid is selectively closed to retain the test swab head in the test swab receiver assembly.

8. The system of claim 1 wherein the test swab retaining lid and test swab receiver are structured so that the test swab head can be disconnected from a test swab stem when the test swab head is positioned in the test swab receiver.

9. The system of claim 1 wherein the test swab receiver assembly comprises a hood that has a concave surface so that the test swab receiver cradles the test swab head.

10. The system of claim 1 wherein the top plate comprises a liquid receiving end that funnels sample-entraining liquid to the immunochromatographic test strip in the base plate.

11. The system of claim 1 wherein the top plate further comprises a flex plate extending downwardly so that the immunochromatographic test strip is sandwiched between the flex plate and at least one raised platform in the base plate-thereby holding the immunochromatographic test strip in position during a test.

12. The system of claim 11 wherein the at least one raised platform comprises a sample pad platform, an intermediate platform, and a terminal platform.

13. The system of claim 12 wherein the immunochromatographic test strip is sandwiched between the flex plate and the terminal platform so that the immunochromatographic test strip is held in position during a test.

14. The system of claim 12 wherein a raised collar is positioned around the sample pad platform, and a raised collar is positioned around the terminal platform.

15. A method of conducting a lateral flow immunoassay test, the method comprising the steps of:
(a) providing the system of claim 1;
(b) initiating a test by collecting a sample on a test swab head;
(c) opening the test swab retaining lid and placing the test swab head in the test swab receiver assembly;
(d) hydrating the test swab head to create the sample-entraining liquid;
(e) allowing the sample-entraining liquid to flow through the swab retention module to the immunochromatographic test strip in the lateral flow cassette module so that a result of the lateral flow immunoassay test is visible through the test results window.

16. The method of claim 15 wherein the system provided in step (a) comprises a swab retention module that includes an outlet port that is sealed with a frangible membrane so that sample-entraining liquid is not allowed (per step (e)) to flow out of the swab retention module, the swab retention module comprising a stand-alone sample-retaining liquid storage container.

17. The method of claim 16 wherein the system provided in step (a), further comprises a top plate having at least one tooth so that when the sample retention module is selectively attached to the lateral flow cassette module, the at least one tooth punctures the frangible membrane and sample-entraining liquid is allowed to flow through the swab retention module to the immunochromatographic test strip per step (e).

18. A method of conducting a lateral flow immunoassay test, the method comprising the steps of:
(a) collecting a sample on a test swab head;
(b) providing a test swab retention module comprising: a test swab receiver assembly having a retaining lid, and a test swab receiver seat, the test swab receiver seat comprising a sealed reservoir;
(c) at least partially inserting the test swab head in the test swab receiver and hydrating the test swab head with a carrier liquid so that sample material is entrained in the liquid and the sample-entraining liquid is retained in the test swab receiver seat reservoir;
(d) fully inserting the test swab head in the test swab receiver and closing the test swab retaining lid so that the test swab head is secure in the test swab receiver and the test swab retaining module comprises a stand-alone sample-retaining liquid storage container;
(e) connecting the test swab retaining module to a lateral flow cassette module so that upwardly oriented teeth on an upper surface of the lateral flow cassette module punctures a seal at a base of the test swab seat reservoir so that sample-entraining liquid flows from the test swab retaining module to the lateral flow cassette module;
(f) exposing an immunochromatographic test strip in the lateral flow cassette module to the sample-entraining liquid so that a result of the lateral flow immunoassay test is visible through a test results window in the lateral flow cassette module.

19. A modular test swab control system comprising:
a test swab retention module configured to hold and retain a test swab head and test sample-entraining liquid;
a lateral flow cassette module selectively attached to the test swab retention module, the lateral flow cassette module comprising:
a top plate having a test results window and a downwardly extending flex plate;
a base plate, and,
a horizontally oriented lateral flow immunoassay immunochromatographic test strip sandwiched between the flex plate and the base plate;
wherein the system is structured so when the test swab head is hydrated, sample-entraining liquid flows downwardly from the test swab head to the immunochromatographic test strip so that a test result is visible through the test results window in the top plate.

20. The system of claim 19 wherein the base plate further comprises at least one raised platform, the immunochromatographic test strip being sandwiched between the flex plate and the raised platform.
